(19) United States
(12) Patent Application Publication
(10) Pub. No.: US 2025/0000999 A1
(43) Pub. Date: Jan. 2, 2025

(54) POLYMERSOMES FOR CLEARANCE OF AMYLOID BETA AND/OR TAU PROTEINS

(71) Applicants: UCL Business Ltd, London (GB); West China Hospital, Sichuan University, Sichuan (CN)

(72) Inventors: Giuseppe BATTAGLIA, London (GB); Diana LEITE, London (GB); Xiaohe TIAN, Sichuan (CN)

(21) Appl. No.: 18/710,264

(22) PCT Filed: Nov. 23, 2022
(86) PCT No.: PCT/GB2022/052970

§ 371 Date: May 15, 2024

(30) Foreign Application Priority Data
Nov. 24, 2021 (WO) PCT/CN2021/132741

(51) Int. Cl. A61K 47/69; A61K 9/127; A61K 47/64; A61P 25/28

(52) U.S. Cl. CPC A61K 47/6915; A61K 9/1273; A61K 47/64; A61P 25/28

(57) ABSTRACT

The present invention is directed to a nanoparticle or microparticle for binding to the surface of an endothelial cell, e.g. a brain endothelial cell, for use in a method for reducing amyloid-β and/or tau levels in an organ (e.g. the brain) of a patient in need thereof, wherein the nanoparticle or microparticle comprises a ligand type on its external surface which is capable of binding to low density lipoprotein receptor-related protein 1 (LRP-1) on said endothelial cell surface, thereby promoting transport of LRP-1 across said endothelial cell. The present invention is further directed to such nanoparticles or microparticles per se which additionally comprise an encapsulated drug selected from an anti-Alzheimer's drug and/or a drug that is useful in reducing amyloid-β and/or tau levels or inhibiting amyloid-β and/or tau formation, and pharmaceutical compositions comprising a plurality of such nanoparticles or microparticles.

### FIELD OF THE INVENTION

The present invention is directed to a nanoparticle or microparticle for binding to the surface of an endothelial cell, e.g. a brain endothelial cell, for use in a method for reducing amyloid-β and/or tau levels in an organ (e.g. the brain) of a patient in need thereof, wherein the nanoparticle or microparticle comprises a ligand type on its external surface which is capable of binding to low density lipoprotein receptor-related protein 1 (LRP-1) on said endothelial cell surface, thereby promoting transport of LRP-1 across said endothelial cell. The present invention is further directed to such nanoparticles or microparticles per se which additionally comprise an encapsulated drug selected from an anti-Alzheimer's drug and/or a drug that is useful in reducing amyloid-β and/or tau levels or inhibiting amyloid-β and/or tau formation, and pharmaceutical compositions comprising a plurality of such nanoparticles or microparticles.

### BACKGROUND TO THE INVENTION

Amyloid-β (Aβ) is a heterogeneous mixture of small peptides (37-43 amino acids) produced by sequential cleavage of amyloid precursor protein (APP). Aβ monomers spontaneously aggregate into neurotoxic aggregates, particularly in the brain, known as oligomers and fibrils. Tau proteins control microtubules in neurons and when they become hyperphosphorylated they assemble into insoluble structure known as neurofibrillary tangles. A faulty transport of Aβ and/or tau proteins across the blood-brain barrier (BBB), and their diminished clearance from the brain, contributes to neurodegenerative and vascular pathologies, including Alzheimer's disease, Parkinson, several dementias, and cerebral angiopathy. At the BBB, Aβ and tau efflux transport is mediated by low-density receptor-related protein (LRP-1). LRP-1 is a multifunctional signalling receptor that binds to a variety of ligands, including Aβ and tau, and directly interacts with them within endothelial cells, e.g. the brain endothelial cells at the BBB, to rapidly initiate their clearance from within organs (e.g. the brain) to the blood. Several studies report that LRP-1 expression declines in brain endothelial cells at the BBB during normal ageing and is further reduced in AD individuals, which favours the accumulation of Aβ and tau in the brain. Hence, strategies to modulate the levels of LRP-1 at the brain blood vessels to restore LRP-1-mediated Aβ and tau clearance from the brain represent a promising approach for the treatment of neurodegenerative disorders, such as Alzheimer's disease.

LRP-1 is a member of the LDL receptor family that plays diverse roles in various biological processes including lipoprotein metabolism, degradation of proteases, activation of lysosomal enzymes and cellular entry of bacterial toxins and viruses. Deletion of the LRP-1 gene leads to lethality in mice, revealing a critical, but as of yet, undefined role in development. Tissue-specific gene deletion studies reveal an important contribution of LRP-1 in the vasculature, central nervous system, in macrophages and in adipocytes.

LRP-1 has been reported to bind to more than 40 ligands, undergoing rapid endocytosis with a half-life of less than 30 seconds (Lillis et al., Physiool. Rev., 2008, 88, 887-918). LRP-1 which has undergone endocytosis can then be trafficked across the endothelial cell via an endolysosomal network, and can subsequently be presented via exocytosis onto the opposite side of the plasma membrane to its original position. This whole process is known as transcytosis. Alternatively, internalised LRP-1 can be marked for degradation in lysosomes.

It would therefore be desirable to develop a medicament which can regulate the expression of LRP-1 in endothelial cells, e.g. brain endothelial cells, in such a way as to maximise LRP-1 mediated clearance of amyloid-β from organs such as the brain. The present invention addresses this problem and provides medicaments that are useful for this purpose.

### SUMMARY OF THE INVENTION

The present inventors have surprisingly discovered that the use of synthetic polymer vesicles (polymersomes) composed of copolymers functionalised with an LRP-1 ligand can suppress degradation of LRP-1 and instead promote the transport of LRP-1 across endothelial cells (e.g. brain endothelial cells) from the apical (blood) to basal (organ, e.g. brain) side via a particular mechanism whereby LRP-1 is transported across the cell by transcytosis, in a structure that is stabilized by syndapin-2. As a result, LRP-1 mediated clearance of amyloid-β and/or tau protein from the basal to apical side of the endothelial cells can be increased.

Moreover, LRP-1 expression levels in the endothelial cells (e.g. brain endothelial cells) were found to be sensitive to structural features of the nanoparticle or microparticle, such as the ligand type and density, the particle surface area, and the steric potential between the nanoparticle or microparticle and the endothelial cell surface. The present invention therefore also provides an algorithm for optimising the nanoparticle or microparticle to provide the highest possible LRP-1 expression levels, and hence most efficient clearance of amyloid-β and/or tau.

Furthermore, nanoparticles or microparticles are a particularly attractive target for activation of LRP-1 transcytosis, because they can be further loaded with relevant drugs to tackle other mechanisms involved in the pathology of relevant diseases caused by, and/or associated with, amyloid beta and/or tau. For example, the nanoparticles or microparticles can be further loaded with relevant drugs to tackle other mechanisms involved in the pathology of Alzheimer's disease, such as inflammation, or cerebral angiopathy. Such nanoparticles or microparticles would allow not only the clearance of amyloid-β and/or tau across the BBB but also the management of other signalling cascades triggered in the brain in neurodegenerative diseases such as Alzheimer's.

The present invention accordingly provides a nanoparticle or microparticle for binding to the surface of an endothelial cell for use in a method for reducing amyloid-β and/or tau levels in an organ of a patient in need thereof, wherein the nanoparticle or microparticle comprises a ligand type on its external surface which is capable of binding to low density lipoprotein receptor-related protein 1 (LRP-1) on said endothelial cell surface, thereby promoting transport of LRP-1 across the endothelial cell. For instance, provided is a nanoparticle or microparticle for binding to the surface of a brain endothelial cell for use in a method for reducing amyloid-β and/or tau levels in the brain of a patient in need thereof, wherein the nanoparticle or microparticle comprises a ligand type on its external surface which is capable of binding to low density lipoprotein receptor-related protein 1 (LRP-1) on said brain endothelial cell surface, thereby promoting transport of LRP-1 across the brain endothelial cell.

The present invention also provides a pharmaceutical composition for use in a method for reducing amyloid-β and/or tau levels in an organ of a patient in need thereof, wherein said pharmaceutical composition comprises a plurality of the nanoparticles or microparticles defined herein, and one or more pharmaceutically acceptable excipients. In a preferred aspect, said organ is the brain.

The present invention also provides a method for reducing amyloid-β and/or tau levels in an organ of a patient in need thereof, wherein said method comprises administration to said patient of a therapeutically effective amount of a nanoparticle or microparticle that comprises a ligand type on its external surface which is capable of binding to low density lipoprotein receptor-related protein 1 (LRP-1) on the surface of an endothelial cell, and thereby promoting transport of LRP-1 across said endothelial cell. In a preferred aspect, the method is a method for reducing amyloid-β and/or tau levels in the brain of a patient in need thereof, wherein said method comprises administration to said patient of a therapeutically effective amount of a nanoparticle or microparticle that comprises a ligand type on its external surface which is capable of binding to low density lipoprotein receptor-related protein 1 (LRP-1) on the surface of a brain endothelial cell, and thereby promoting transport of LRP-1 across said brain endothelial cell.

The present invention also provides the use of a nanoparticle or microparticle for the manufacture of a medicament for reducing amyloid-β and/or tau levels in an organ of a patient in need thereof, wherein said nanoparticle or microparticle comprises a ligand type on its external surface which is capable of binding to low density lipoprotein receptor-related protein 1 (LRP-1) on the surface of an endothelial cell, and thereby promoting transport of LRP-1 across said endothelial cell. In a preferred aspect, the organ is the brain and the endothelial cell is a brain endothelial cell.

The present invention also provides a nanoparticle or microparticle for binding to the surface of an endothelial cell comprising:
(i) a ligand type on its external surface which is capable of binding to low density lipoprotein receptor-related protein 1 (LRP-1) on the surface of an endothelial cell, thereby promoting transport of LRP-1 across said endothelial cell; and
(ii) an encapsulated drug selected from an anti-Alzheimer's drug and/or a drug that is useful in reducing amyloid-β and/or tau levels or inhibiting amyloid-β and/or tau formation, preferably wherein said drug is selected from donepezil, galantamine, rivastigmine and memantine.

In a preferred aspect, the endothelial cell is a brain endothelial cell.

The present invention also provides a pharmaceutical composition comprising a plurality of the nanoparticles or microparticles according to the invention, and one or more pharmaceutically acceptable excipients.

### BRIEF DESCRIPTION OF THE FIGURES

FIG. 1 shows the particle size distribution measured by dynamic light scattering for the AP₂₂-POs (a) and a transmission electron micrograph of the AP₂₂-POs (staining agent: phosphotungstic acid (PTA)) (b).

FIG. 2 is a box plot showing the expression of LRP-1 in polarised mouse brain endothelial cells, normalised to loading control (GAPDH), both before and after being treated with AP₂₂-POs for 2 hours. *=P<0.05, Student's T-test (n=6).

FIG. 3 shows the basal to apical transport of amyloid-β across polarised brain endothelial cells pre-treated with AP₂₂-POs for 2 hours, wherein the AP₂₂-POs are applied to either the apical (top circle at each time point) or basal (bottom circle at each time point) side of the membrane. Data are presented as mean±standard deviation.

FIG. 4 shows the effect of polymersome administration to Alzheimer's diseased mice (groups 1-3) and healthy mice (groups 4-5) on the levels of amyloid-β and tau proteins. (A) shows a Western blot for amyloid-β and tau proteins, in addition to actin as a control, for each cohort of mice. (B) represents the amyloid-β concentration in each cohort in graphical format. (C) represents the tau concentration in each cohort in graphical format. n=3 for all cohorts.

FIG. 5 shows the effect of polymersome administration to Alzheimer's diseased mice (groups 1-3) and healthy mice (groups 4-5) on the levels of liver function markers ALT (A), AST (B) and ALP (C). n=3 for all cohorts.

FIG. 6 shows the effect of polymersome administration to Alzheimer's diseased mice (groups 1-3) and healthy mice (groups 4-5) on the levels of kidney function markers BUN (A), Cr (B) and ALP (C). n=3 for all cohorts.

FIG. 7 shows the concentration of amyloid beta and tau in the blood plasma over time, after administration of polymersomes to Alzheimer's diseased mice.

FIG. 8 shows PET/CT scans of APP-PS1 Alzheimer model mice (top), APP-PS1 Alzheimer model mice treated with polymersomes (middle) and healthy mice (bottom), all injected with [18F](E)-4-(2-(6-(2-(2-(2-18F-fluoroethoxy)ethoxy)ethoxy)pyridin-3-yl)vinyl)-N-methylbenzamine to label amyloid beta. The scans show a significant reduction in the amount of amyloid beta present in the brain in the group of animals that was treated with polymersomes.

FIG. 9 shows a heat map of the total ligand/LRP-1 binding energy in brain endothelial cells as a function of LRP-1 receptor density, nanoparticle/microparticle radius and ligand number. The black/darkest region of the graph indicates a stronger than optimal affinity of the nanoparticles/microparticles to the target cells, resulting primarily in endocytosis of the LRP-1 receptors in the brain endothelial cells. The grey region of the graph indicates a weaker than optimal binding of the nanoparticles/microparticles to the target cells. The white/lightest region of the graph indicates the optimal level of nanoparticle/microparticle binding to LRP-1 on the surface of the brain endothelial cells which promotes transcytosis of the LRP-1/nanoparticle or microparticle complex across the brain endothelial cell, and subsequent upregulation of LRP-1.

### DETAILED DESCRIPTION

### Nanoparticles and Microparticles

The nanoparticles and microparticles for use in the present invention can be any nanoparticles or microparticles suitable for delivery of a drug cargo to a target site of action in vivo. A “nanoparticle”, as defined herein, is any particle from 1 to 100 nm in size. A “microparticle”, as defined herein, is any particle greater than 0.1 μm and up to 100 μm in size. Suitable nanoparticles or microparticles for use in the present invention include polymersomes, liposomes, synthosomes, latex, micelles, nanocrystals, quantum dots, metallic nanoparticles, oxide nanoparticles, silica nanoparticles, protein cages, nano- and micro-gels, dendrimers, virus-like particles, protein, polymers or any other colloidal materials that fall within the aforementioned size range. Typically, however, the nanoparticles or microparticles for use in the present invention are polymersomes, liposomes, synthosomes or micelles. Typically, the nanoparticles or microparticles are self-assembled structures.

The nanoparticles and microparticles of the present invention may be of any feasible geometry, e.g. substantially spherical, ellipsoidal, cylindrical or bilayer form, but typically they are substantially spherical. Thus, a substantially spherical nanoparticle for use in the present invention has a (largest) diameter of from 1 to 100 nm, and a substantially spherical microparticle for use in the present invention has a (largest) diameter greater than 0.1 μm and up to 100 μm. Typically, a (largest) diameter of a nanoparticle or microparticle of the present invention is in the range 50 to 5000 nm. More typically, the diameter is in the range 50 to 1000 nm. Typically, the nanoparticles or microparticles for use in the present invention have a number average diameter of less than 300 nm, preferably less than 250 nm, most preferably less than 200 nm or 150 nm. In one aspect, the nanoparticle or microparticle for use in the present invention is a nanoparticle. Alternatively, the nanoparticle or microparticle for use in the present invention is a microparticle. Typically, particle size is measured using transmission electron microscopy (TEM). Typically, particle size distribution is measured using dynamic light scattering (DLS).

Preferably, the nanoparticle or microparticle for use in the invention is a polymersome. Polymersomes are synthetic vesicles formed from amphiphilic block copolymers. Examples of polymersomes are described in US 2010/0003336 A1, WO 2017/144849, WO 2017/158382, WO 2017/199023, WO 2017/191444, WO 2019/197834, WO 2020/144467 and WO 2020/225538, the contents of each of which are herein incorporated by reference in their entirety. Over the last fifteen years they have attracted significant research attention as versatile carriers because of their colloidal stability, tuneable membrane properties and ability in encapsulating or integrating other molecules (for one representative review article, see Lee and Feijen, J Control Release, 2012, 161(2), 473-83, the contents of which are herein incorporated by reference in their entirety).

Polymersomes are typically self-assembled structures. Polymersomes typically comprise an amphiphilic block copolymer, i.e. a block copolymer that comprises a hydrophilic block and a hydrophobic block. For example, the polymersome may comprise at least two such amphiphilic block copolymers, which are different from one another.

Such copolymers are able to mimic biological phospholipids. Molecular weights of these polymers are much higher than naturally-occurring phospholipid-based surfactants such that they can assemble into more entangled membranes (Battaglia and Ryan, J. Am. Chem. Soc., 2005, 127, 8757-8764, the contents of which are herein incorporated by reference in their entirety), providing a final structure with improved mechanical properties and colloidal stability. Furthermore, the flexible nature of the copolymer synthesis allows the application of different compositions and functionalities over a wide range of molecular weights and consequently of membrane thicknesses. Thus the use of these block copolymers as delivery vehicles offers significant advantages.

Polymersomes are often substantially spherical. Polymersomes typically comprise an amphiphilic membrane. The membrane is generally formed from two monolayers of amphiphilic molecules, which align and entangle to form an enclosed core with hydrophilic head groups facing the core and the exterior of the vesicle, and hydrophilic tail groups forming the interior of the membrane.

The thickness of the bilayer is generally between 2 and 100 nm, more typically between 2 and 50 nm (for instance between 5 and 20 nm). These dimensions can routinely be measured, for example by using transmission electron microscopy (TEM) and/or and small angle X-ray scattering (SAXS) (see, for example, Battaglia and Ryan, J. Am. Chem. Soc., 2005, 127, 8757-8764, the contents of which are herein incorporated by reference in their entirety).

When a polymersome is formed from more than one different type of copolymer, different regions of the polymersome typically have different bilayer thicknesses. For example, if a polymersome is formed from two different types of copolymer, preferably the thickness of the polymersome bilayer of a first region is from 1 to 10 nm, more preferably from 2 to 5 nm. Preferably the thickness of the polymersome bilayer of a second region is from 5 to 50 nm, for instance from 10 to 40 nm. More preferably the thickness of the polymersome bilayer of the second region is from 5 to 20 nm. Preferably the thickness of the polymersome bilayer of the first region is less than the thickness of the polymersome bilayer of the second region. Alternatively, the copolymers can have same thickness but different chemical compositions, which in turn create two different permeabilities with one copolymer forming a bilayer which is less permeable than the other.

In aqueous solution, normally an equilibrium exists between different types of structures, for instance between polymersomes and micelles. It is preferred that at least 80 wt %, more preferably at least 90 wt % or 95 wt % and most preferably all of the structures in solution are present as polymersomes. This can be achieved using the methods outlined herein.

It is known that when two different polymersome-forming copolymers are mixed to form a hybrid vesicle they phase-separate and thus give rise to polymersomes that contain discrete regions corresponding to the discrete copolymers. For example, this phenomenon is described in detail in LoPresti et al., ACS NANO, 2011, 5(3), 1775-1784, the contents of which are herein incorporated by reference in their entirety. Polymersomes can be readily manufactured by applying these known synthetic principles.

A polymersome is preferably capable of dissociating and releasing the encapsulated drug once it has reached the tissue of interest (i.e. the target tissue). Non-limiting, exemplary tissues of interest are discussed in more detail later and include cells (e.g. CNS cells) beyond the blood-brain barrier. Preferably the polymersome is capable of dissociating and releasing the encapsulated drug after it has been internalised, via endocytosis, within a target cell (e.g. a CNS cell). Preferably therefore, the polymersome is configured to bind to, and cross, brain endothelial cells which make up the blood-brain barrier.

Dissociation may be promoted by a variety of mechanisms, such as pH sensitivity of the block copolymer, thermal sensitivity of the block copolymer, hydrolysis (i.e. water sensitivity of the block copolymer) and/or redox sensitivity of the block copolymer.

The hydrophobic block of a copolymer comprised in the polymersome may also comprise pendant cationisable moieties as pendant groups. Cationisable moieties are, for instance, primary, secondary or tertiary amines as well as imidazole groups, capable of being protonated at pHs below a value in the range 3 to 6.9. Alternatively the group may be a phosphine.

Preferably, the hydrophobic block of the polymersome has a degree of polymerisation of at least 50, more preferably at least 70. Preferably, the degree of polymerisation of the hydrophobic block is no more than 250, even more preferably, no more than 200. Typically, the degree of polymerisation of the hydrophilic block is at least 10, preferably at least 15, and more preferably at least 20. It is preferred that the ratio of the degree of polymerisation of the hydrophilic to hydrophobic block is in the range 1:2.5 to 1:8. All of these limitations promote polymersome, rather than micelle, formation.

The hydrophilic block may be based on condensation polymers, such as polyesters, polyamides, polyanhydrides, polyurethanes, polyethers (including polyalkylene glycols, especially polyethylene glycol (PEG)), polyimines, polypeptides, polypeptoids, polyureas, polyacetals and polysaccharides. Preferably, the hydrophilic block is based on a polymer selected from a poly(alkylene glycol), poly(vinyl pyrrolidone) (PVP), poly(2-methacryloyloxyethyl phosphorylcholine) (PMPC), poly(glycerol)s, poly(amino acid)s, polysarcosine, poly(2-oxazoline)s, poly[oligo(ethylene glycol) methyl methacrylate] and poly(N-(2-hydroxypropyl)methacrylamide). Most preferably, the hydrophilic block is based on PEG, poly(propylene glycol) or poly[oligo(ethylene glycol) methyl methacrylate]. The hydrophilic block may have zwitterionic pendant groups, in which case the zwitterionic pendant groups may be present in the monomers and remain unchanged in the polymerisation process. It is alternatively possible to derivatise a functional pendant group of a monomer to render it zwitterionic after polymerisation.

In one embodiment of this invention, the monomer from which the hydrophobic block is formed is 2-(diisopropylamino)ethyl methacrylate (DPA) or 2-(diethylamino)ethyl methacrylate (DEA).

In another embodiment, the hydrophobic block is formed from 2-(diisopropylamino)ethyl methacrylate (DPA) or 2-(diethylamino)ethyl methacrylate (DEA) and the hydrophilic block is based on a polyester, polyamide, polyanhydride, polyurethane, polyether, polyimine, polypeptide, polypeptoid, polyurea, polyacetal or polysaccharide. Preferably, the hydrophobic block is formed from 2-(diisopropylamino)ethyl methacrylate (DPA) or 2-(diethylamino)ethyl methacrylate (DEA) and the hydrophilic block is based on PEG, poly(propylene glycol) or poly[oligo(ethylene glycol) methyl methacrylate]. More preferably, a polymersome for use in the present invention comprises di-block PEG-PDPA, wherein PEG is poly(ethylene glycol), and the PDPA is poly(2-(diisopropylamino)ethyl methacrylate). Alternatively, a polymersome for use in the present invention comprises di-block POEGMA-PDPA, wherein POEGMA is poly[oligo(ethylene glycol) methyl methacrylate], and the PDPA is poly(2-(diisopropylamino)ethyl methacrylate). A particularly preferred diblock copolymer is (P[(OEG)₁₀MA₂₀]-PDPA₁₀₀). These copolymers have the ability to self-assemble in water or PBS and create vesicles having an aqueous lumen into which drugs can be loaded. The PEG functionality provides pendant hydroxyl groups, which act as handles for easy/reliable functionalisation of the polymers with ligands (as discussed below), while avoiding protein opsonization (giving polymersomes long circulation time and low unspecific binding). PDPA, meanwhile, is a pH-sensitive block that triggers the disassembly of polymersomes at pH values below 6.4, which is a typical pH during early stage endocytosis. The pH-sensitivity allows the drug payload to be released in the cell cytosol, upon internalization of the polymersome within a cell.

The block copolymer may be a simple A-B block copolymer, or may be an A-B-A or B-A-B block linear triblock copolymer or a (A)₂B or A(B)₂ star copolymers (where A is the hydrophilic block and B is the hydrophobic block). It may also be an A-B-C, A-C-B or B-A-C block linear triblock copolymers or a ABC star copolymers (blocks linked together by the same end), where C is a different type of block. C blocks may, for instance, comprise functional, e.g. cross-linking or ionic groups, to allow for reactions of the copolymer, for instance in the novel compositions. Crosslinking reactions especially of A-C-B type copolymers, may confer useful stability on polymersomes. Cross-linking may be covalent, or sometimes, electrostatic in nature. Cross-linking may involve addition of a separate reagent to link functional groups, such as using a difunctional alkylating agent to link two amino groups. The block copolymer may alternatively be a star type molecule with hydrophilic or hydrophobic core, or may be a comb polymer having a hydrophilic backbone (block) and hydrophobic pendant blocks or vice versa. Such polymers may be formed for instance by the random copolymerisation of monounsaturated macromers and monomers.

The microparticle or nanoparticle (e.g. polymersome) may also contain a moiety provided on the its surface which creates an interference steric potential with the surface of the target cell, such as a polymer brush. Without wishing to be bound by any particular theory, for the highest levels of selectivity of polymersome binding to the desired target to be observed, each ligand on the surface of the nanoparticle or microparticle individually should have a very low binding affinity for its target receptor. In practice, selective ligands with such a low binding energy to a target receptor are not readily available. Thus, in some embodiments of the present invention, the nanoparticle or microparticle comprises a polymer brush on its external surface, in order to create a steric potential to mitigate the strength of binding of the ligand(s) to the target receptor(s).

Typically a polymer brush comprises a naturally occurring polymer, such as a polypeptide or polysaccharide, or a synthetic polymer, such as any of the amphiphilic block copolymers described above. Components on the external surface of the target cell, such as glycans, glycoproteins and glycolipids (collectively referred to as the “glycocalyx”), are also believed to contribute to this repulsive steric potential. Preferred polymeric components of the polymer brush include poly(ethylene glycol) (PEG), poly(vinyl pyrrolidone) (PVP), poly(2-methacryloyloxyethyl phosphorylcholine) (PMPC), poly(glycerol)s, poly(sulfobetaine), poly(carboxybetaine), poly(amino acid)s, polysarcosine, poly(2-oxazoline)s, poly(N-(2-hydroxypropyl)methacrylamide), polyglycols, heparin, dextran, poly(ethylene glycol)-poly(2-(diisopropylamino)ethyl methacrylate) and/or poly(oligo(ethylene glycol) methyl ether methacrylate) (POEGMA).

Preferably, the polymer brush has a degree of polymerisation of at least 5, more preferably at least 10. Preferably, the degree of polymerisation of the polymer brush is no more than 500, e.g. no more than 300, or no more than 200. Preferably, the polymer brush has a length of from 1.5 to 350 nm, and more preferably from 3 to 210 nm.

Polymersomes of the present invention may comprise any of the structural and/or functional features of the polymersomes described in any of WO 2017/144849, WO 2017/158382, WO 2017/199023, WO 2017/191444, WO 2019/197834, WO 2020/144467 and WO 2020/225538, the contents of each of which are herein incorporated by reference in their entirety.

Further details of a suitable process for polymerising the monomers are to be found in WO 03/074090, the contents of which are herein incorporated by reference in their entirety.

Exemplary methods that can be used for polymerising the monomers are atom-transfer radical polymerisation (ATRP) (see, e.g., an exemplary method described in Du et al., J. Am. Chem. Soc., 2005, 127, 17982-17983), living radical polymerisation process, functional NCA (N-carboxyanhydride) polymerisation with efficient postpolymerization modification and ring opening polymerisation (ROP). Living radical polymerisation has been found to provide polymers of monomers having a polydispersity (of molecular weight) of less than 1.5, as judged by gel permeation chromatography. Polydispersities in the range of from 1.2 to 1.4 for the or each block are preferred. The polymersomes may be loaded using a pH change system, electroporation or film hydration. In a pH change system process, polymer is dispersed in aqueous liquid in ionized form, in which it solubilises at relatively high concentrations without forming polymersomes. Subsequently the pH is changed such that some or all of the ionized groups become deprotonated so that they are in non-ionic form. At the second pH, the hydrophobicity of the block increases and polymersomes are formed spontaneously.

A method of forming polymersomes with an encapsulated material (e.g. an encapsulated drug) in the core may involve the following steps: (i) dispersing the amphiphilic copolymer in an aqueous medium; (ii) acidifying the composition formed in step (i); (iii) adding the material to be encapsulated to the acidified composition; and (iv) raising the pH to around neutral to encapsulate the material.

This method preferably comprises a preliminary step wherein the amphiphilic copolymer is dispersed in an organic solvent in a reaction vessel and the solvent is then evaporated to form a film on the inside of the reaction vessel.

Step (ii), of acidifying the composition, typically reduces the pH to a value below the pKa of the pendant group.

Another method of forming polymersomes with an encapsulated material in the core may involve the following steps: (i) dispersing the amphiphilic copolymer, and when needed the material to be encapsulated, in an organic solvent (e.g. a 2:1 chloroform:methanol mixture) in a reaction vessel; (ii) evaporating the solvent to form a film on the inside of the reaction vessel; and (iii) re-hydrating the film with an aqueous solution, optionally comprising a solubilized material to be encapsulated.

Another method of forming polymersomes with an encapsulated material in the core may involve the following steps: (i) dispersing the amphiphilic copolymer, and when needed the material to be encapsulated, in an organic solvent in a reaction vessel; (ii) adding the aqueous solvent to enable solvent switch and the formation of polymersomes on the inside of the reaction vessel; and (iii) optionally electroporating the obtained polymersomes to allow encapsulation of water-soluble bioactive molecules.

UV spectroscopy and HPLC chromatography may be used to calculate the encapsulation efficiency, using techniques well known in the art. An alternative method for forming polymersomes with an encapsulated material may involve simple electroporation of the material and polymer vesicles in water. For instance the drug may be contacted in solid form with an aqueous dispersion of polymer vesicles and an electric field applied to allow the formation of pores on the polymersomes membrane. The solubilized material molecules may then enter the polymersome vesicles though the pores. This is followed by membrane self-healing process with the consecutive entrapment of the material molecules inside the polymersomes.

Alternatively, material dissolved in organic solvent may be emulsified into an aqueous dispersion of polymer vesicles, whereby solvent and the material become incorporated into the core of the vesicles, followed by evaporation of solvent from the system.

The polymersomes used in the invention may be formed from two or more different block copolymers. In this embodiment, in the method of forming polymersomes, a mixture of the two or more block copolymers is used.

For example, 0.01% to 10% (w/w) of material to be encapsulated is mixed with copolymer in the methods described above.

Alternatively, the nanoparticle or microparticle for use in the present invention may be a liposome. A liposome is a spherical vesicle having at least one lipid bilayer. Typically, a liposome comprises a phospholipid, e.g. phosphatidylcholine, but may also include other lipids, such as egg phosphatidylethanolamine, so long as they are compatible with a lipid bilayer structure. The major types of liposomes include the multilamellar vesicle (MLV, with several lamellar phase lipid bilayers), the small unilamellar liposome vesicle (SUV, with one lipid bilayer), the large unilamellar vesicle (LUV), and the cochleate vesicle.

Typically, the liposomes are fusogenic liposomes. This means that they are capable of fusing with a membrane, e.g. the cell surface membrane of a target cell, or the membrane of an endosome within the cell. Fusion of the bilayer of a fusogenic liposome with the cell surface membrane results in the incorporation of the liposome bilayer into the cell surface membrane, and the release of the drug cargo contained within the lysosome into the cell cytosol. Alternatively, the liposome may be internalized within a target cell via endocytosis, and the drug cargo carried within the liposome is released after fusion of the liposome bilayer with the endosomal membrane. The pH within an endosome is slightly acidic and therefore it is advantageous for the liposomes to be pH sensitive, e.g. the stability of the liposome structure is decreased at lower pH, facilitating fusion with the endosomal membrane. Other environments having low pH can also trigger the fusion of such liposomes, e.g., the low pH found in tumors or sites of inflammation.

Liposomes may be zwitterionic structures. Alternatively, liposomes may be amphoteric liposomes. This means that the liposomes have an isoelectric point and are negatively charged at higher pH values and positively charged at lower pH values. Typical pH-responsive elements in pH-sensitive liposomes include cholesterol hemisuccinate (CHEMS), palmitoylhomocysteine, dioleoylglycerol hemisuccinate (DOG-Succ) and the like.

Alternatively, the nanoparticle or microparticle for use in the present invention may be a synthosome. Synthosomes are a particular type of polymersome engineered to contain channels (transmembrane proteins) that selectively allow certain chemicals to pass through the membrane, into or out of the vesicle.

Alternatively, the nanoparticle or microparticle for use in the present invention may be a micelle. Micelles are aggregates (or supramolecular assemblies) of molecules having both hydrophilic and hydrophobic regions, dispersed in a liquid. Typically in an aqueous solution, the aggregated micelle is arranged such that the hydrophobic regions of the molecules are sequestered in the centre of the micelle, whilst the hydrophilic regions of the molecules present on the external surface of the micelle, and contact the aqueous solvent. Typically, micelles are substantially spherical in shape, although other shapes such as ellipsoid, cylindrical, torus and discoid are also possible.

Alternatively, the nanoparticle or microparticle for use in the present invention may be any object able to encapsulate and/or conjugate any type of bioactive molecules, such as anticancer drugs, proteins, peptides (natural or not), antibodies, fragment of antibodies, dyes, and the like.

### Targeting Ligands for LRP-1

The nanoparticle or microparticle comprises a ligand type on its external surface which is capable of binding to low density lipoprotein receptor-related protein 1 (LRP-1). A “ligand” may also be referred to herein as a “targeting moiety”. By “on its external surface” is meant that each ligand is located such that it is able to interact with its target (as opposed to being located at an inaccessible position that precludes interaction with the target, for example by being encapsulated within the nanoparticle or microparticle).

As discussed above, LRP-1 is a receptor that is highly expressed on the brain endothelial cells that form the blood-brain barrier. Thus, in a particularly preferred embodiment the nanoparticle or microparticle for use in the present invention is configured to bind to the surface of a brain endothelial cell. Typically the ligand binds selectively to LRP-1, i.e. to the exclusion of any significant level of binding to other proteins.

In one embodiment, the ligand is a moiety that is attached to the external surface of the nanoparticle or microparticle. Examples of suitable ligands include antibodies, antibody fragments, aptamers, oligonucleotides, small molecules, peptides and carbohydrates. Peptide, protein, antibody and antibody fragment ligands are particularly preferred. Peptides that bind to the receptor LRP-1 are known in the art. For example, Angiochem (Montreal, Canada) have developed peptides that the leverage the LRP-1 mediated pathway to cross the blood-brain barrier when conjugated to drug cargos. One specific example of a peptide that is suitable for use in the present invention is Angiopep-2, which is a peptide having the sequence TFFYGGSRGKRNNFKTEEY. Further examples of suitable targeting moieties are disclosed in WO 2013/078562, the contents of which are herein incorporated by reference in their entirety (and, specifically, the ligand peptides disclosed in which are herein incorporated by reference). However, any such moiety can be used as a ligand in the present invention. The suitability of any given moiety to target LRP-1 can be determined using routine assay methods, involving testing for the ability of the moiety to bind specifically to the receptor.

It has been found that provision of a nanoparticle or microparticle that features a ligand that targets the LRP-1 receptor enables increased clearance of amyloid-β from the basal (brain) to apical (blood) side of the brain endothelial cells, resulting in a neuroprotective effect.

Without wishing to be bound by any particular theory, it is believed that the neuroprotective effect is a result of the transport of LRP-1 across the brain endothelial cell, from the apical side to the basal side. The mechanism of LRP-1 transport is believed to occur via transcytosis. This process typically comprises the following stages: (i) binding of the LRP-1 ligand to LRP-1 on the surface of the brain endothelial cell on the apical side, (ii) internalization of LRP-1 (preferably, as part of an LRP-1/nanoparticle or LRP-1/microparticle complex) into a vesicular carrier within the brain endothelial cell by endocytosis, (iii) transport (or “trafficking”) of LRP-1 (preferably, the LRP-1/nanoparticle or LRP-1/microparticle complex) across the brain endothelial cell, (iv) presentation of the transported LRP-1 (preferably, the LRP-1/nanoparticle or LRP-1/microparticle complex) on the basal side membrane of the brain endothelial cell via exocytosis (and in the case of an LRP-1/nanoparticle or LRP-1/microparticle complex, the nanoparticle or microparticle may then dissociate from the complex). A similar mechanism of LRP-1 transport is believed to be operative on any other endothelial cells in which LRP-1 is expressed.

It is believed that in the transport stage (iii) of this process, LRP-1 (or LRP-1/nanoparticle or LRP-1/microparticle) transport is mediated by a structure that is stabilized by syndapin-2; confocal laser scanning microscopy studies show that LRP-1 and syndapin-2 are co-localized during transport. Said structure is typically tubular, or substantially tubular, in shape. It has been found that the binding of a nanoparticle or microparticle as described herein to LRP-1 receptors on the endothelial cells can promote this syndapin-2-mediated transcytosis mechanism, resulting in transport of the nanoparticle or microparticle and the LRP-1 receptor from one surface of the endothelial cell to the opposing surface (i.e. from the apical to basal side).

Thus, in some embodiments the present invention provides a nanoparticle or microparticle as defined herein for use in a method for reducing amyloid-β and/or tau levels in an organ (e.g. the brain) of a patient in need thereof, wherein said method comprises the binding of the nanoparticle or nanoparticle to an LRP-1 receptor on the surface of an endothelial cell (e.g. a brain endothelial cell), and the subsequent transport of LRP-1 (or, an LRP-1/nanoparticle or LRP-1/microparticle complex) across the endothelial cell. Preferably, the nanoparticle or nanoparticle binds to an LRP-1 receptor on the apical surface of the endothelial cell. Preferably, the transport of LRP-1 (or, the LRP-1/nanoparticle or LRP-1/microparticle complex) across the endothelial cell occurs via transcytosis. More preferably, the transport of LRP-1 (or, the LRP-1/nanoparticle or LRP-1/microparticle complex) across the endothelial cell is mediated via a structure that is stabilized by syndapin-2. Preferably, following transport of LRP-1 (or, the LRP-1/nanoparticle or LRP-1/microparticle complex) across the endothelial cell, LRP-1 (or, the LRP-1/nanoparticle or LRP-1/microparticle complex) is presented on the basal surface of the endothelial cell. Preferably, in the case of transport of an LRP-1/nanoparticle or LRP-1/microparticle complex, the microparticle or nanoparticle then dissociates from the LRP-1/nanoparticle or LRP-1/microparticle complex. Most preferably, the endothelial cell is a brain endothelial cell.

Thus, in some embodiments the present invention provides a method for reducing amyloid-β and/or tau levels in an organ (e.g. the brain) of a patient in need thereof, wherein said method comprises administration to said patient of a therapeutically effective amount of a nanoparticle or microparticle that comprises a ligand type on its external surface which is capable of binding to low density lipoprotein receptor-related protein 1 (LRP-1), and wherein the method further comprises binding of the nanoparticle or microparticle to LRP-1 on the surface of an endothelial cell (e.g. a brain endothelial cell), and subsequent transport of LRP-1 (or, an LRP-1/nanoparticle or LRP-1/microparticle complex) across the endothelial cell. Preferably, the nanoparticle or nanoparticle binds to an LRP-1 receptor on the apical surface of an endothelial cell. Preferably, the transport of LRP-1 (or, the LRP-1/nanoparticle or LRP-1/microparticle complex) across the endothelial cell occurs via transcytosis. More preferably, the transport of LRP-1 (or, the LRP-1/nanoparticle or LRP-1/microparticle complex) across the endothelial cell is mediated via a structure that is stabilized by syndapin-2. Preferably, following transport of LRP-1 (or, the LRP-1/nanoparticle or LRP-1/microparticle complex) across the endothelial cell, LRP-1 (or, the LRP-1/nanoparticle or LRP-1/microparticle complex) is presented on the basal surface of the endothelial cell. Preferably, in the case of transport of an LRP-1/nanoparticle or LRP-1/microparticle complex, the microparticle or nanoparticle then dissociates from the LRP-1/nanoparticle or LRP-1/microparticle complex. Most preferably, the endothelial cell is a brain endothelial cell.

Furthermore, it has been found that the promotion of the transcytosis mechanism is associated with an increase in LRP-1 expression within the endothelial cells. The promotion of transcytosis and upregulation in LRP-1 expression in this manner enables an accumulation of LRP-1 on the basal side of the endothelial cells, which is thought to be advantageous in the clearance of both amyloid-β and tau proteins from the organ (e.g. the brain).

Thus, in some embodiments the present invention provides a nanoparticle or microparticle as defined herein for use in a method for reducing amyloid-β and/or tau levels in an organ (e.g. the brain) of a patient in need thereof, wherein said method comprises the binding of the nanoparticle or nanoparticle to an LRP-1 receptor on the surface of an endothelial cell (e.g. a brain endothelial cell), and further comprises an increase in the expression of LRP-1 in the endothelial cell.

Thus, in some embodiments the present invention provides a method for reducing amyloid-β and/or tau levels in the brain of a patient in need thereof, wherein said method comprises administration to said patient of a therapeutically effective amount of a nanoparticle or microparticle that comprises a ligand type on its external surface which is capable of binding to low density lipoprotein receptor-related protein 1 (LRP-1), and wherein the method further comprises binding of the nanoparticle or microparticle to LRP-1 on the surface of an endothelial cell (e.g. a brain endothelial cell), and further comprises an increase in the expression of LRP-1 in the endothelial cell.

Moreover, it is believed that structural features of the nanoparticle or microparticle can influence the extent to which the mechanism of LRP-1 transcytosis is promoted. In particular, the present inventors have found that the correlation between the avidity of the nanoparticle or microparticle and the promotion of transcytosis is non-linear. As used herein, the term “avidity” refers to the accumulated strength of multiple affinities of individual ligand-receptor interactions. Thus, a polymersome comprising many ligands on its external surface which bind to LRP-1 will typically have a higher avidity than a polymersome of the same dimensions comprising relatively fewer such ligands on its external surface, as a greater total number of ligand-receptor interactions are possible. A polymersome comprising a more potent LRP-1 binding ligand on its external surface would also be anticipated to have a higher avidity than a corresponding polymersome comprising a less potent LRP-1 binding ligand on its external surface. As avidity increases from a low level to a higher level, transcytosis of LRP-1 is promoted; however, as avidity continues to increase to a yet higher level, the observed amount of transcytosis of LRP-1 decreases again. It is thought that high avidity nanoparticles or microparticles instead promote disintegration of LRP-1 instead of transcytosis.

There is therefore a “sweet spot” in avidity of the nanoparticle or microparticle for use in the present invention, in order to maximize transcytosis of LRP-1 from the apical to basal side of the endothelial cells.

The avidity of the nanoparticle/microparticle-LRP-1 interaction is therefore a relevant parameter in optimal nanoparticle/microparticle design for achieving the greatest level of the desired pharmacological effects. The present inventors have developed an empirical formula for determining an optimal nanoparticle/microparticle avidity. Specifically, the number of the ligand type on the external surface of the microparticle or nanoparticle (A) is preferably such that the properties of the microparticle or nanoparticle satisfy the following relationship:

$$ln [ {( 1 + λ {Ae}^{- β ( {ϵ}_{B} + {U}_{S} )} )}^{ζ} - 1 ] ∈ [ 20 , 40 ]$$

wherein:
λ is the density of the ligand type on the external surface of the microparticle or nanoparticle (number per nm²
A is the microparticle or nanoparticle surface area (in nm²);
ξ is the number of the LRP-1 receptors accessible to the nanoparticles, and thus ξ=ΓA where, Γ is the LRP-1 surface density (number per nm²) and A is as defined above;
β=(k_{B}T)⁻¹ wherein k_{B} is the Boltzmann constant (in JK⁻¹) and T is the absolute temperature in Kelvin;
ε_{B} is the single energy of binding of a ligand type/LRP-1 receptor pair (in J); and
u_{S} is the steric potential between the nanoparticle or microparticle and the cell surface (in J).

For the avoidance of doubt, in the above relationship the symbol ϵ means that the value of the formula lies between the two integers in square brackets, i.e. between 20 and 40.

The relevant parameters in the formula can be readily determined by a person skilled in the art using e.g. the methods described below.

The number (and density) of each type of ligand on the external surface of a polymersome can typically be controlled during synthesis of the polymersome by varying the ratio of ligand-bound copolymer and “pristine” copolymer (i.e. diblock copolymer that does not have a ligand attached). For any given system, the number of ligands per polymersome is then given by the copolymer self-assembly parameter (related to the polymer molecular weight and the packing factor) and the polymersome size. The number of each type of ligand on the external surface of a polymersome (and hence the density of receptors) can typically be verified using mass spectrometry.

The surface area of the nanoparticle or microparticle can be measured by several microscopic techniques, including transmission electron microscopy, scanning electron microscopy, atomic force microscopy and similar, as well as by scattering techniques such as dynamic or static light scattering. Preferably, the surface area is measured by transmission electron microscopy.

The single ligand binding potential ε_{B} between a given ligand and the LRP-1 receptor can be measured experimentally by binding assays including Surface plasmon resonance spectroscopy, Isothermal titration calorimetry, radiolabeling or fluorescent labelling. It can also be estimated computationally using molecular docking and or molecular dynamics methods.

The steric potential u_{S} can be calculated as u_{S}=u_{P}+u_{G}, i.e. the sum of the steric potential arising from the glycocalyx brush on the cell surface (u_{P}) and the steric potential arising from the polymer brush that coats the nanoparticle (u_{G}). The magnitude of u_{P} and u_{G} depends on how accessible the ligands and receptor are. Their values can be derived as follows:

$$β {u}_{G} = \frac{4 π {R}^{3}}{3 {( {σ}_{GAG} )}^{\frac{3}{2}}} {( 1 - {δ}_{G}^{2} )}^{\frac{9}{4}} and β {u}_{P} = \frac{{V}_{LRP 1}}{{( {σ}_{P} )}^{\frac{3}{2}}} {( 1 - {δ}_{P}^{2} )}^{\frac{9}{4}}$$

where:
β is as defined above;
R is the radius of the nanoparticle (in nm), which can typically be determined using the same microscopic technique as for the determination of surface area above;
δ_{G} is the ratio between the average glycocalyx thickness, h_{G}=d_{PG}+b_{GAG}\sqrt{N_{GAG}}(where d_{PG} is the average length of proteoglycans, which estimated from structure available within the protein database is typically around 4.5 nm, b_{GAG} is the Khun length of the GAG chains and is typically 7 nm, and N_{GAG} is the degree of polymerisation of the GAG chains typically between 80 and 100) and the extracellular LRP-1 length which can readily be obtained from structural biology databases known in the art and is typically 60 nm;
δP is the ratio between the polymer length that stabilises the nanoparticle surface, h_{P}, and the ligand polymer tether; both values are fixed by the design of the nanoparticle;

$${V}_{LRP 1} = \frac{1}{6} π {r}_{L}^{3}$$

and is the volume of the LRP-1 segment engaged with the ligand and changes with ligand type, with r_{L} being the distance between the ligand binding site and the tip of the LRP-1 and which can be readily determined by a person skilled in the art using molecular modelling techniques; for Angiopep-2 this is estimated from molecular modelling at about 3.5 nm;
σ_{GAG} is the surface area occupied by a single GAG chain, on the cell surface; and
σ_{P} is the surface area occupied by a single polymer chain on nanoparticle and it is defined a priori during the nanoparticle design.

The LRP-1 surface density (number per nm²) Γ, and the surface area occupied by a single GAG chain, σ_{GAG}, can both be determined by a binding assay to endothelial cells (e.g. brain endothelial cells) in vitro using well defined and homogenous nanoparticles decorated with variable ligand numbers. An example procedure for measuring LRP-1 expression levels is provided in Example 1 below.

This formula therefore provides a useful and novel empirical tool for determining the optimum number of LRP-1 ligands on the external surface of a nanoparticle or microparticle for use in the present invention.

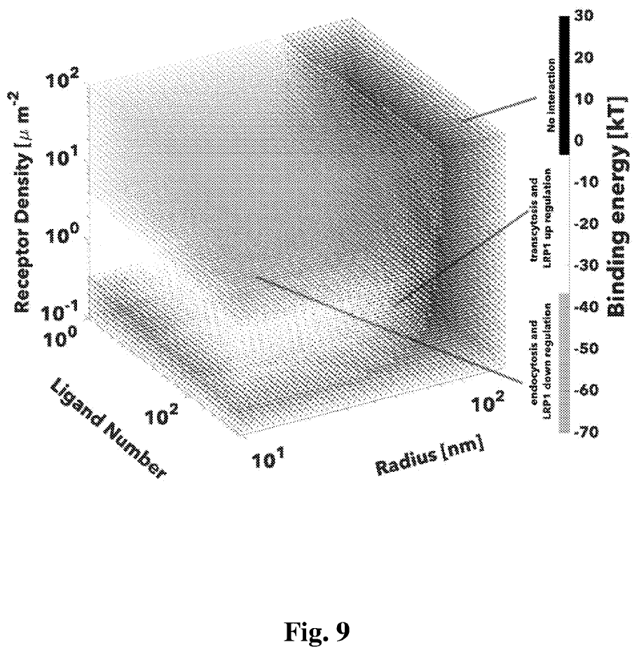

A plot showing the effects of LRP-1 receptor density, polymersome radius and ligand number (i.e. the key parameters in the above formula that can be influenced by polymersome design and the system being targeted) on the total LRP-1/nanoparticle or microparticle binding energy in brain endothelial cells is shown in FIG. 9. The black/darkest region of the graph indicates a stronger than optimal affinity of the polymersomes to the target cells, resulting primarily in endocytosis of the LRP-1 receptors in the brain endothelial cells. The grey region of the graph indicates a weaker than optimal binding of the polymersomes to the target cells. The white/lightest region of the graph indicates the optimal level of polymersome binding to LRP-1 on the surface of the brain endothelial cells which promotes transcytosis of the LRP-1/polymersome complex across the brain endothelial cell, and subsequent upregulation of LRP-1. This is the region corresponding to systems for which the value of the formula above lies between 20 and 40. Thus, it is apparent that there is a “sweet spot” in total LRP-1/nanoparticle or microparticle binding energy, achievable via smart nanoparticle or microparticle design, which leads to the most effective operation of the LRP-1 transcytosis mechanism and upregulation that ultimately drives amyloid beta and tau clearance from the brain.

Typically, the nanoparticle or microparticle comprises from 2 to 1000 ligands of the ligand type that binds to LRP-1, preferably from 5 to 500 ligands of the ligand type, more preferably from 10 to 200 ligands of the ligand type, yet more preferably from 15 to 100 ligands of the ligand type, and most preferably from 20 to 50 ligands of the ligand type.

Typically, the LRP-1 ligand is attached to a polymer component on the external surface of the nanoparticle or microparticle. In the case of polymersomes, the ligand is typically attached to the hydrophilic block of the amphiphilic diblock copolymer.

A ligand can be attached to the external surface of the nanoparticle or microparticle using routine techniques, for example by adapting well known methods for attaching ligands to polymers, drugs, nucleic acids, antibodies and other substances. The attachment may be non-covalent (e.g. electrostatic) or covalent, though it is preferably covalent. For example, when the nanoparticle or microparticle is a polymersome, the targeting moiety can be attached by reacting a suitable functional group on the targeting moiety (including but not limited to an amine group, a carboxyl group and a thiol group) with a corresponding functional group on at least one of the copolymers that form, or will form, the polymersome. The attachment can be effected either before the polymersome structure is formed from the copolymers, or after the polymersomes have been formed.

In a particularly preferred embodiment, the nanoparticle or microparticle is a polymersome which comprises, on its external surface, a polymer brush comprising poly(ethylene glycol)-poly(2-(diisopropylamino)ethyl methacrylate) and each ligand type. Thus, the ligands are inserted in the polymer brush of polymersomes made of poly(ethylene glycol)-poly(2-(diisopropylamino)ethyl methacrylate), typically by employing a solvent-switch method. Typically, the density of the ligands within the brush can also be varied.

It is also possible to provide for attachment of the ligand to the copolymers by first chemically activating either or both of the ligand and the copolymers. For example, a peptide ligand may be activated by adding a reactive species to one of its termini, such as a cysteine moiety (whose thiol group is well known to react readily with functional groups such as the widely used maleimide moiety). Similarly, a copolymer can be activated by functionalising it with a reactive species (e.g. a maleimide moiety when the targeting moiety carries a thiol group). The copolymer may be provided with such a reactive species either by functionalisation of the copolymer itself, or by providing suitable monomers prior to the polymerisation that forms the copolymer, or by providing a suitable initiator for the polymerisation.

In a particularly preferred embodiment, the nanoparticle or microparticle is a polymersome wherein one or more ligands on the external surface of the polymersome are covalently bound to a poly(ethylene glycol) molecule. Tethering of the ligands to PEG molecules of different chain lengths in this way enables control over the deepness of the ligand insertion within the polymer brush. This in turn affects the steric repulsive potential, u_{S}, between the ligand and the target cell surface receptor. As discussed above, this steric potential is an important factor in determining the optimum number of ligands on the surface of the nanoparticle or microparticle for binding to a particular cell type.

A ligand may be attached directly to the external surface of the nanoparticle or microparticle, or alternatively it may be attached via a chemical spacer.

When the nanoparticle or microparticle is a polymersome, a ligand may also be a pendant group of a polymer comprised by the polymersome (i.e. at least one of the copolymers forming the polymersome itself). Clearly in this embodiment it is not necessary to undertake separate synthetic steps to attach the ligand to the copolymer or the resulting polymersome.

Suitable pendant groups generally include any group that corresponds to a ligand as defined elsewhere herein. In one illustrative embodiment, the targeting moiety is a phosphorylcholine moiety, i.e. a group having the formula

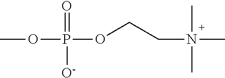

A phosphorylcholine moiety is a zwitterionic moiety that can constitute a pendant group in one or more of the monomers that form the copolymers comprised in a polymersome.

The phosphorylcholine moiety selectively targets scavenger receptor class B, member 1 (SCARB1) over-expressed by macrophages and other immune cells; in particular it enables a polymersome featuring phosphorylcholine moieties to enter such cells.

In some embodiments, the nanoparticle or microparticle for use in the present invention may comprise more than one ligand types on its external surface that is targeted to LRP-1. Thus, the nanoparticle or microparticle may comprise two, three, four, five or more such ligand combinations.

The binding of the nanoparticle or microparticle to LRP-1 also enables the nanoparticle or microparticle itself to cross the BBB. Thus, any encapsulated drug within the nanoparticle or microparticle can be effectively delivered into both the CNS parenchyma and CNS cells. In particular, it has been found that the endothelial transcytosis mechanism does not involve acidification of the nanoparticle or microparticle in membrane-trafficking organelles, which is important to avoid premature disintegration of the polymersome and concomitant release of the encapsulated drug. Still further, the LRP-1 receptor is associated with traditional endocytosis in CNS cells, which, subsequent to navigation across the BBB, aids the delivery of the drug within their cytosol (via disintegration of the nanoparticle or microparticle).

### Further Targeting Ligands

In some embodiments, the nanoparticle or microparticle for use in the present invention may also comprise at least one further ligand type on its external surface that binds to a different complementary receptor, in addition to the ligand type(s) that bind(s) to LRP-1. Thus, the nanoparticle or microparticle may comprise a second ligand type that is capable of binding to a second receptor type on the endothelial cell (e.g. brain endothelial cell) surface. The nanoparticle or microparticle may also comprise a third, fourth, fifth or more ligand type that is capable of binding to a third, fourth, fifth etc. receptor type on the endothelial cell (e.g. brain endothelial cell) surface. In one embodiment, the nanoparticle or microparticle therefore comprises from two to seven different ligand types on its external surface, each of which is capable of binding to a complementary receptor type on the cell surface. Preferably in this embodiment, the nanoparticle or microparticle of the present invention comprises from two to six different ligand types on its external surface, more preferably from three to five different ligand types, and most preferably four different ligand types. Thus, the nanoparticle or microparticle comprises from one to five further ligand types on its external surface in addition to the ligand targeted to LRP-1. Preferably the nanoparticle or microparticle comprises from two to four further ligand types, and most preferably three further ligand types.

Typically in this embodiment, the nanoparticle or microparticle comprises from 2 to 1000 ligands of the second ligand type. Preferably, the nanoparticle or microparticle comprises from 5 to 1000 ligands of the second ligand type, more preferably from 10 to 500 ligands of the second ligand type, even more preferably from 20 to 200 ligands of the second ligand type, and most preferably from 50 to 100 ligands of the second ligand type.

Typically in this embodiment, the nanoparticle or microparticle comprises from 2 to 1000 ligands of a subsequent (i.e. third or higher order) ligand type. Preferably, the nanoparticle or microparticle comprises from 5 to 1000 ligands of the subsequent ligand type, more preferably from 10 to 500 ligands of the subsequent ligand type, even more preferably from 20 to 200 ligands of the subsequent ligand type, and most preferably from 50 to 100 ligands of the subsequent ligand type.

Typically, the combination of ligands on the surface of the nanoparticle or microparticle leads to a total binding energy of from 8k_{B}T to 30k_{B}T, where k_{B} is Boltzmann's constant and T is the temperature. Without wishing to be bound by any particular theory, this is thought to lead to on-off association profiles of the nanoparticles or microparticles wherein the receptors are saturated only above a given onset receptor density, whilst the nanoparticles or microparticles do not bind at all at lower receptor densities.

Each ligand type is adapted to enable the nanoparticle or microparticle to bind to a target. Typically the ligand binds selectively to the target. The target is a chemical substance that is located on or in the vicinity of the tissue of interest (and thus enables the nanoparticle or microparticle to accumulate specifically at the tissue of interest in preference to other sites). The target is preferably a receptor, e.g. a receptor that is present in particularly high quantity at the target tissue of interest. Most preferably, the target is a receptor on or within a cell surface membrane.

Each ligand type can be any ligand that binds specifically to the target. As is well known in the art, for example from the well-developed field of bioconjugates, a wide range of substances can be used as ligands, e.g. to target receptors.

In one embodiment, each ligand is a moiety that is attached to the external surface of the nanoparticle or microparticle. Examples of suitable ligands include antibodies, antibody fragments, aptamers, oligonucleotides, small molecules, peptides and carbohydrates. Peptide, protein, antibody and antibody fragment ligands are particularly preferred. However, any such moiety can be used as a ligand in the present invention. The suitability of any given moiety to target any given receptor can be determined using routine assay methods, involving testing for the ability of the moiety to bind specifically to the receptor.

Without wishing to be bound by any particular theory, it is believed that the multiplexing of ligands on the surface of a nanoparticle or microparticle in this fashion confers the property of “super-selectivity” for the target cells. This concept is discussed in detail in WO 2020/225538, the contents of which are herein incorporated by reference in their entirety. In short, the principle behind “super-selectivity” is that if multiple different ligand types are present on the surface of a nanoparticle or microparticle scaffold, the selectivity of the nanoparticles/microparticles for their target cell populations is very high, leaving other cells untouched.

Hence, it is believed that polymersomes functionalized with two, or more, ligand types, each having relatively low affinity for their target receptor, can avoid targeting undesired cells, but still bind effectively to the target cells. Moreover, mutations in cell surface receptors will less likely lead to evasion of detection by the nanoparticles or microparticles.

Examples of other receptors that are highly expressed on the endothelial cells that form the blood-brain barrier (in addition to LRP-1), which might be targeted by a second (or higher order) ligand on the external surface of the nanoparticle or microparticle, include scavenger receptor class B, member 1 (SCARB1), a transferrin receptor (TFRC), folate receptor 1 (FOLR1) and epidermal growth factor receptor (EGFR).

In one embodiment, the nanoparticle or microparticle contains a further ligand type, and preferably one ligand type, that targets the SCARB1 receptor. The protein encoded by this gene is a plasma membrane receptor for high density lipoprotein cholesterol (HDL) that facilitates the uptake of cholesterol esters from circulating lipoproteins. Additional findings suggest a critical role for SCARB1 in cholesterol metabolism, signalling, motility, and proliferation of cancer cells and thus a potential major impact in carcinogenesis and metastasis. Malignant tumours display remarkable heterogeneity to the extent that even at the same tissue site different types of cells with varying genetic background may be found. In contrast, SCARB1 has been found to be consistently overexpressed by most tumour cells (e.g. HeLa and FaDu cells). Recent findings indicate that the level of SCARB1 expression correlate with aggressiveness and poor survival in certain cancers. SCARB1 is also a receptor for hepatitis C virus glycoprotein E2.

Ligands that bind to SCARB1 are known in the art. One such ligand is poly(2-(methacryloyloxy)ethyl phosphorylcholine) (PMPC).

In one embodiment, therefore, one ligand type on the nanoparticle or microparticle scaffold targets LRP-1 and another ligand type on the scaffold targets SCARB1.

In another embodiment, the nanoparticle or microparticle contains a further ligand type, and preferably one ligand type, that targets TFRC. This gene encodes a cell surface receptor necessary for cellular iron uptake by the process of receptor-mediated endocytosis. This receptor is required for erythropoiesis and neurologic development.

Iron as an important element plays crucial roles in various physiological and pathological processes. Iron metabolism behaves in systemic and cellular two levels that usually are in balance conditions. The disorders of the iron metabolism balances relate with many kinds of diseases including Alzheimer's disease, osteoporosis and various cancers. In systemic iron metabolism that is regulated by hepcidin-ferroportin axis, plasma iron is bound with transferrin (TF) which has two high-affinity binding sites for ferric iron. The generic cellular iron metabolism consists of iron intake, utilization and efflux. During the iron intake process in generic cells, transferrin receptors (TFRs) act as the most important receptor mediated controls. TFR1 and TFR2 are two subtypes of TFRs those bind with iron-transferrin complex to facilitate iron into cells. TFR1 is ubiquitously expressed on the surfaces of generic cells, whereas TFR2 is specially expressed in liver cells. TFR1 has attracted more attention than TFR2 by having diverse functions in both invertebrates and vertebrates. Recently reports showed that TFR1 involved in many kinds of diseases including anaemia, neurodegenerative diseases and cancers. Most importantly, TFR1 has been verified to be abnormally expressed in various cancers. Thus, TFR1 is postulated as a potential molecular target for diagnosis and treatment for cancer therapy.

In one embodiment, therefore, one ligand type on the nanoparticle or microparticle scaffold targets LRP-1 and another ligand type on the scaffold targets TFRC.

In another embodiment, the nanoparticle or microparticle contains a further ligand type, and preferably one ligand type, that targets FOLR1. The protein encoded by this gene is a member of the folate receptor family. Members of this gene family bind folic acid and its reduced derivatives, and transport 5-methyltetrahydrofolate into cells. This gene product is a secreted protein that either anchors to membranes via a glycosyl-phosphatidylinositol linkage or exists in a soluble form. Mutations in this gene have been associated with neurodegeneration due to cerebral folate transport deficiency.

The folate cycle sustains key metabolic reactions and is essential for rapidly growing cells. Under physiologic conditions, exogenous reduced folates (water-soluble B vitamins) are predominantly transported into cells via the low-affinity, high-capacity, ubiquitously expressed reduced folate carrier (RFC; bidirectional anion-exchange mechanism). Once in the cell, folates play an essential role in the biosynthesis of purines and thymidine, which in turn are required for DNA synthesis, methylation, and repair. Folates are also transported by high-affinity FRs. In humans, there are four isoforms of the FR (FRα, FRβ, FRγ, and FRδ). FRα, FRβ, and FRδ are attached to the cell surface by a glycosylphosphatidylinositol anchor, while FRγ is a secreted protein. Because FRα is expressed on the cell surface in a tumour-specific manner, it provides the potential to allow not only tumour localization, but also selected delivery of therapeutic agents to the malignant tissue, minimizing collateral toxic side-effects.

There are a number of unique advantages to exploiting FR as a diagnostic and therapeutic target. First, FRα is located on the luminal surface of epithelial cells in most proliferating nontumor tissues and is inaccessible to circulation. In contrast, FRα is expressed all over the cell in malignant tissue and is accessible via circulation. Second, FR has the ability to bind to folic acid, a relatively innocuous, small molecule that can rapidly penetrate solid tumours and is amenable to chemical conjugation with other molecules. Once a folate conjugate is bound to FR, it is internalized into the cell and the FRα is rapidly recycled to the cell surface via the FR-mediated endocytic pathway. These factors all emphasize the potential role of FRα in the diagnosis and treatment of specific tumour types.

In one embodiment, therefore, one ligand type on the nanoparticle or microparticle scaffold targets LRP-1 and another ligand type on the scaffold targets FOLR1.

In another embodiment, the nanoparticle or microparticle contains a further ligand type, and preferably one ligand type, that targets EGFR. The protein encoded by this gene is a transmembrane glycoprotein that is a member of the protein kinase superfamily. This protein is a receptor for members of the epidermal growth factor family. EGFR is a cell surface protein that binds to epidermal growth factor. Binding of the protein to a ligand induces receptor dimerization and tyrosine autophosphorylation and leads to cell proliferation.

Epidermal growth factor receptors (EGFRs) are a large family of receptor tyrosine kinases (TK) expressed in several types of cancer, including breast, lung, esophageal, and head and neck. EGFR and its family members are the major contributors of a complex signaling cascade that modulates growth, signaling, differentiation, adhesion, migration and survival of cancer cells. EGFR binds to its cognate ligand EGF, which further induces tyrosine phosphorylation and receptor dimerization with other family members leading to enhanced uncontrolled proliferation. Due to their multi-dimensional role in the progression of cancer, EGFR and its family members have emerged as attractive candidates for anti-cancer therapy.

Specifically, the aberrant activity of EGFR has shown to play a key role in the development and growth of tumor cells, where it is involved in numerous cellular responses including proliferation and apoptosis. The epidermal growth factor receptor (EGFR) signalling pathway is also a strong contender for both initiating and determining clinical outcomes in many respiratory diseases. Deregulation of the EGFR pathway causing aberrant EGFR signalling is associated with the early stage pathogenesis of lung fibrosis, cancer and numerous airway hypersecretory diseases, including COPD, asthma and cystic fibrosis.

In one embodiment, therefore, one ligand type on the nanoparticle or microparticle scaffold targets LRP-1 and another ligand type on the scaffold targets EGFR.

Ligands for binding to each of these receptor are well known in the art. Example ligands for LRP-1 and SCARB1 are discussed above. Example ligands for TFRCs, e.g. TFR1, are transferrin and transferrin mimic peptide. An example ligand for FOLR1 is folic acid. An example ligand for EGFR is the peptide YHWYGYTPQNVI peptide.

### Encapsulated Drug

The nanoparticle or microparticle for use in the present invention may optionally comprise a drug encapsulated within the nanoparticle or microparticle. For the avoidance of doubt, it is also possible to encapsulate a plurality of different drugs within a single nanoparticle or microparticle, or to provide a plurality of nanoparticles or microparticles each containing a particular encapsulated drug.

As will be readily understood, the encapsulated drug is selected in accordance with the disorder to be treated. Non-limiting examples of such disorders are described elsewhere in this disclosure.

Typically, the encapsulated drug is selected from an anti-Alzheimer's drug, a drug for treating cerebral angiopathy and/or a drug that is useful in reducing amyloid-β and/or tau levels or inhibiting amyloid-β and/or tau formation. Thus, in some embodiments, the encapsulated drug is an anti-Alzheimer's drug. In other embodiments, the encapsulated drug is a drug for treating cerebral angiopathy. In other embodiments, the encapsulated drug is a drug that is useful in reducing amyloid-β and/or tau levels. In other embodiments, the encapsulated drug is a drug that is useful in inhibiting amyloid-β and/or tau formation.

Non-limiting examples of such drugs include donepezil, galantamine, rivastigmine, memantine, and combinations thereof.

### Pharmaceutical Compositions

The nanoparticle or microparticle of the present invention can be formulated as a pharmaceutical composition using routine techniques known in the art. For example, pharmaceutical compositions already utilized for the formulation of nanoparticles or microparticles such as polymersomes or drug-containing liposomes.

The pharmaceutical composition comprises a plurality of the nanoparticles or microparticles of the present invention. It also comprises one or more pharmaceutically acceptable excipients. The one or more pharmaceutically acceptable excipients may be any suitable excipients. The pharmaceutical composition is typically aqueous, i.e. it contains water (in particular sterile water). Common pharmaceutical excipients include lubricating agents, thickening agents, wetting agents, emulsifying agents, suspending agents, preserving agents, fillers, diluents, binders, preservatives and adsorption enhancers, e.g. surface penetrating agents. Solubilizing and/or stabilizing agents may also be used, e.g. cyclodextrins (CD). A person skilled in the art will be able to select suitable excipients based on their purpose. Common excipients that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe fur Pharmazie, Kosmetik und angrenzende Gebiete (Edition Cantor Aulendorf, 1989), the contents of both of which are incorporated by reference herein in their entirety).

A typical pH of the aqueous pharmaceutical composition is 7.0 to 7.6, preferably 7.2 to 7.4. Pharmaceutically acceptable buffers may be used to achieve the required pH. The pharmaceutical composition may be in the form of a sterile, aqueous, isotonic saline solutions.

Pharmaceutical compositions of the invention may be administered to a patient by any one or more of the following routes: oral, systemic (e.g. transdermal, intranasal, transmucosal or by suppository), or parenteral (e.g. intramuscular, intravenous or subcutaneous). Compositions of the invention can take the form of tablets, pills, capsules, semisolids, powders, sustained release formulations, solutions, suspensions, elixirs, aerosols, transdermal patches, bioadhesive films, or any other appropriate compositions. Typically, though, the pharmaceutical composition is an injectable composition, e.g. it is suitable for parental administration, and preferably it is suitable for intravenous delivery, for example by infusion.

### Medical Uses of the Nanoparticles or Microparticles

The nanoparticles or microparticles of the present invention are able to target tissues including, but not limited to cells (e.g. CNS cells) beyond the blood-brain barrier, and to promote basal to apical clearance of amyloid-β and tau proteins from the brain. As discussed above, the high efficiency in targeting emerges, at least in part, through the presence of an appropriate number of ligands (i.e. targeting moieties) targeted to the receptor LRP-1 on the external surface of the nanoparticle or microparticle (e.g. as part of the polymers themselves or as distinct moieties attached thereto).

Thus, the present invention provides nanoparticles or microparticles as defined herein for use in a method for reducing amyloid-β and/or tau levels in a patient in need thereof. Thus, in one embodiment the nanoparticles or microparticles are for use in a method for reducing amyloid-β and/or tau levels in a patient. The patient is typically a mammal, more typically a human patient. Amyloid-β and/or tau may be removed from any organ or tissue in which high levels have accumulated. Preferably, the organ from which amyloid-β and/or tau may be removed is the brain. Examples of other organs from which amyloid-β and/or tau may be removed include the heart and the kidney.

Particular conditions that can be treated by the nanoparticles and microparticles described herein include Alzheimer's disease and cerebral angiopathy.

Thus, in some embodiments the present invention provides a nanoparticle or a microparticle as defined herein, for use in a method of treating or preventing Alzheimer's disease in a patient.

In other embodiments, the present invention provides a nanoparticle or a microparticle as defined herein, for use in a method of treating or preventing cerebral angiopathy in a patient.

The present invention also provides a method of reducing amyloid-β and/or tau levels or inhibiting amyloid-β in the brain, wherein said method comprises administration to said patient of a therapeutically effective amount of a nanoparticle or microparticle as described herein to said patient.

In some embodiments, the present invention provides a method of treating or preventing Alzheimer's disease in a patient in need thereof, wherein said method comprises administration to said patient of a therapeutically effective amount of a nanoparticle or microparticle as described herein to said patient.

In other embodiments, the present invention provides a method of treating or preventing cerebral angiopathy in a patient in need thereof, wherein said method comprises administration to said patient of a therapeutically effective amount of a nanoparticle or microparticle as described herein to said patient.

The present invention also provides use of a nanoparticle or microparticle as described herein for the manufacture of a medicament for reducing amyloid-β and/or tau levels in the brain of a patient in need thereof.

In some embodiments, the present invention provides use of a nanoparticle or microparticle as described herein for the manufacture of a medicament for the treatment or prevention of Alzheimer's disease in a patient in need thereof.

In other embodiments, the present invention provides use of a nanoparticle or microparticle as described herein for the manufacture of a medicament for the treatment or prevention of cerebral angiopathy in a patient in need thereof.

In some embodiments, the nanoparticles and microparticles of the invention which comprise an encapsulated drug selected from an anti-Alzheimer's drug and/or a drug that is useful in reducing amyloid-β and/or tau levels or inhibiting amyloid-β and/or tau formation are for use in a method for reducing amyloid-β and/or tau levels, or inhibiting amyloid-β and/or tau formation, in a patient in need thereof. The activity of such nanoparticles and microparticles typically result from both (i) the effect of the polymersomes on LRP-1 trafficking and expression in brain endothelial cells, and (ii) the encapsulated drug, which is released at its target site within the brain. As will be readily understood, the encapsulated drug is selected in accordance with the disease to be treated. Preferably, the encapsulated drug is selected from donepezil, galantamine, rivastigmine and memantine.

Medical uses and methods of treatment, of course, involve the administration of a therapeutically effective amount of the nanoparticle or microparticle. A therapeutically effective amount of the nanoparticles or microparticles is administered to a patient. As used herein, the term “therapeutically effective amount” refers to an amount of the biologically active molecule which is sufficient to reduce or ameliorate the severity, duration, progression, or onset of a disorder being treated, prevent the advancement of a disorder being treated, cause the regression of, prevent the recurrence, development, onset or progression of a symptom associated with a disorder being treated, or enhance or improve the prophylactic or therapeutic effect(s) of another therapy. The precise amount of biologically active molecule administered to a patient will depend on the type and severity of the disease or condition and on the characteristics of the patient, such as general health, age, sex, body weight and tolerance to drugs. It will also depend on the degree, severity and type of the disorder being treated. The skilled artisan will be able to determine appropriate dosages depending on these and other factors. A typical dose, however, is from 0.001 to 1000 mg, measured as a weight of the drug, according to the activity of the specific drug, the age, weight and conditions of the subject to be treated, the type and severity of the disease and the frequency and route of administration. Preferably, daily dosage levels are from 0.001 mg to 4000 mg.

The nanoparticles, microparticles or pharmaceutical compositions comprising such nanoparticles or microparticles may be administered to the patient by any suitable method. Preferably, however, the nanoparticles or microparticles are administered parenterally (e.g. by intramuscular, intravenous or subcutaneous injection). Most preferably, the nanoparticles or microparticles are administered by injection.

### EXAMPLES

### Example 1: Effect of Polymersomes with LRP-1 Targeting Ligands on LRP-1 Expression and Amyloid-β Clearance from the Brain

Polymersomes that are each functionalised with 22 Angiopep-2 ligands on their external surface (AP₂₂-POs) were synthesised.

Synthetic vesicles were made using amphiphilic copolymers made by poly(ethylene glycol) (PEG) as a hydrophobic block and poly(2-(diisopropylamino)ethyl methacrylate) (PDPA) as a hydrophilic block.

P[(OEG)₁₀MA]₂₀-PDPA₁₀₀, Cy5-P[(OEG)₁₀MA]₂₀-PDPA₁₀₀, Angiopep-P[(OEG)₁₀MA]₂₀-PDPA₁₀₀ and PMPC₂₅-PDPA₇₀ copolymers were synthesised as reported in Tian et al., Sci Rep, 2015, 5, 11990, the contents of which are incorporated herein by reference in their entirety.

The Angiopep-2 peptides on the surface of the polymersome target the LRP-1 receptor and the PMPC ligands target the SCARB1 receptor. About 5% of the POEGMA-PDPA chains were labelled with Cy5 dye to allow fluorescence quantification. The Angiopep peptide was conjugated to POEGMA-PDPA copolymers and these were mixed at different concentration with pristine POEGMA-PDPA. The resulting arrangement of peptide expressed on the surface and immersed in the oligoethylene oxide chain (N_{P}⁼10). The PMPC chains were co-polymerised with DPA to form PMPC₂₄-PDPA₇₀ and these were mixed with pristine POEGMA-PDPA chains at different concentrations.

To make 10 mg/mL polymersomes, the amount of copolymers was weighed and dissolved using pH 2 PBS. Once the film dissolved the pH was increased to 5.0. Peptide-functionalised copolymers were then added, in order to avoid acidic degradation. The pH was gradually increased to pH 6.8-7.0, eventually stopping at pH 7.4-7.5. Polymersomes formed during prolonged stirring at pH 6.8-7.0. The polymersomes were then ultrasound sonicated for 15-30 mins, at 4° C. The purification of polymersomes was finally performed by passing through a gel permeation chromatography column pre packed with Sepharose 4B (Sigma Aldrich). For long-term storage, the polymersomes can be kept at 4° C. and when conjugated to dyes are protected from light. The peptide-functionalised polymersomes were freshly made just before use. In this regard, it is important to note that although POEGMA-PDPA and PMPC-PDPA chains can undergo phase separation forming patchy polymersomes (see LoPresti et al., ACS Nano, 2011, 5(3), 1775-1784), the cellular experiments were performed right after preparation and hence without giving the sufficient time to separate (3-5 days).

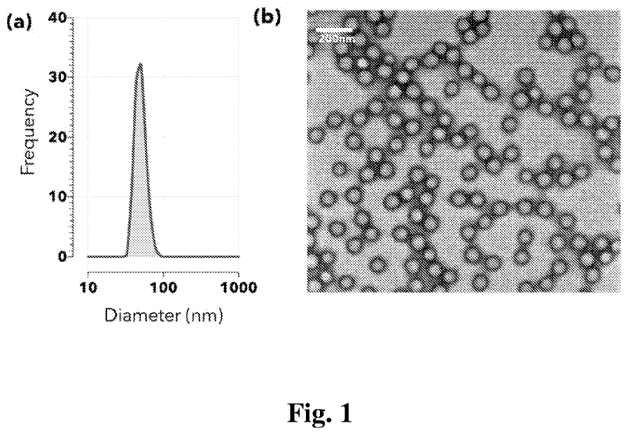

The particle size distribution of the polymersomes was measured via dynamic light scattering (DLS) (see FIG. 1(a)). All the formulations had an average radius of 40 nm (+/−10 nm) and the addition of the ligand did not alter the final structure as confirmed by both TEM and DLS. The polymersomes were further characterised by transmission electron microscopy (JEOL 2100) using phosphotungstenic acid as staining agent and dynamic light scattering (Malvern Nanosizer) (see FIG. 1(b)). LRP-1 expression was measured by Western blot (WB) and immunofluorescence (IF). For WB cells were washed twice with PBS, and RIPA buffer containing protease inhibitors (1:50) was added directly to the membranes and left on ice for 1 hour. Cells were collected and centrifuged, and the supernatant was collected for WB analysis. Protein levels in the cell lysates were determined using the BCA Protein Assay Kit. Lysates were mixed with Laemmli sample buffer, and proteins (10 μg) were separated on 10% SDS polyacrylamide gels and transferred to polyvinylidene difluoride membranes. Membranes were blocked with 5% (w/v) nonfat milk in tris-buffered saline (TBS) containing 0.1% (w/v) Tween 20 (TBS-T) for 1 hour and then incubated with a rabbit monoclonal antibody to LRP-1 overnight at 4° C. After washing with TBS-T, the membranes were incubated with a secondary antibody for 2 hours at room temperature and imaged using Odyssey CLx (LI-COR Biosciences). The membranes were further probed for glyceraldehyde-3-phosphate dehydrogenase (GAPDH) as a loading control. For IF Coronal brain sections were obtained from animals. Briefly, brain sections were incubated in 20% (v/v) normal horse serum in PBS containing 0.3% (w/v) Triton X-100 for 2 hours at room temperature under gentle agitation followed by incubation with primary antibody anti-syndapin-2 overnight at 4° C. Sections were washed with PBS, incubated with the corresponding secondary antibody and FITC-conjugated lectin (1:200) for 2 hours, and washed with PBS. Brain sections were mounted on glass slides in Vectashield Mounting Media.

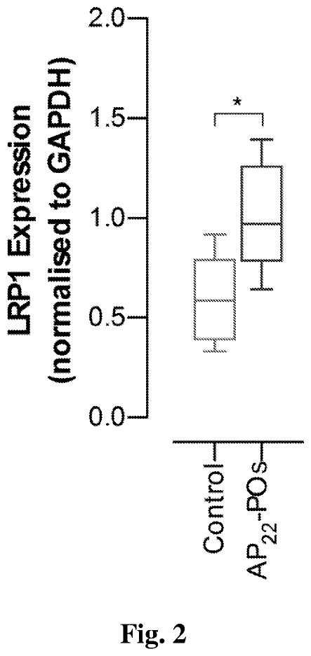

FIG. 2 shows a box plot of LRP-1 expression levels in the control sample of brain endothelial cells and the sample of brain endothelial cells that have been treated with the AP₂₂-POs. It can be observed that there is a significant increase in the LRP-1 expression level in the cells after treatment with the polymersomes.

In a further experiment, brain endothelial cells were pre-treated for 2 hours with AP₂₂-POs that were applied to either the apical (blood) or basal (brain) side of the cells in the Transwell. Subsequently, the basal to apical transport of amyloid-β using the Ab40 as model was measured for 4 hours. The permeability of amyloid-β was normalised to untreated cells.

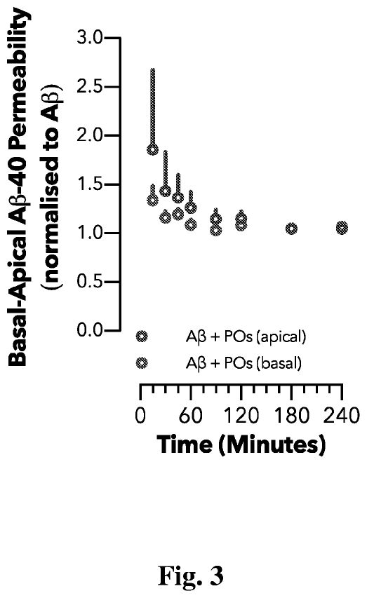

FIG. 3 shows the results of this experiment. It is notable that the higher levels of basal to apical transport of amyloid-β was observed in the cells that were pre-treated with AP₂₂-POs on the apical side rather than on the basal side. This suggests that the polymersomes per se are not directly responsible for triggering amyloid-β clearance from the brain tissue, but instead supports an indirect mechanism of action (i.e. the increasing polymersome levels on the apical side of the cells promotes LRP-1 transcytosis and upregulation).

In this example, each polymersome has 22 Angiopep-2 ligands with λ=1.1×10⁻³, ε_{B}=−15 k_{B}T, the polymersome radius is 40 nm and surface area is 20.11×10³ nm², the polymersome is made of POEGMA with a δ_{P}=0.1 insertion parameter allowing V_{LRP1}=270 nm³ of LRP-1 inserting within the POEGMA brush and σ_{P}⁻¹=0.08, targeting cells expressing LRP-1 with Γ=1.5×10⁻⁴ proteins per nm² that corresponds to ξ=3 with δ_{G}=0.9 and a glycocalyx density of σ_{GAG}⁻¹=0.0088 glycosaminoglycan (GAG) chains per nm² giving rise to a total steric potential u_{S}=11.24 k_{B}T. These polymersomes therefore fulfil the mathematical relationship provided in the description, as the value of the formula 1n[(1+λAe^{-β(εB+uS)})^{ξ}−1] is 20.6.

### Example 2: In Vivo Studies in Normal and Diseased Mice

The AP₂₂-POs synthesised in Example 1 were also administered intravenously to mice and the in vivo effects on amyloid-β and tau levels were monitored. As a control, effects on liver and kidney function were also investigated. Five different groups of mice (n=3) were utilised in the study, as follows:

| | | | Experiment | |
|---|---|---|---|---|
| Group | Healthy or diseased | Composition administered | time | |
| 1 | Alzheimer's disease | 200 μL PBS | 1 | hour |
| | model mouse | | | |
| 2 | Alzheimer's disease | 200 μL polymersomes | 1 | hour |
| | model mouse | (1 mg/mL) | | |
| 3 | Alzheimer's disease | 1200 μL polymersomes, | 24 | hours |
| | model mouse | dosed at 50 μL/hour | | |
| 4 | Healthy mouse | None | N/A | |
| 5 | Healthy mouse | 200 μL polymersomes | 1 | hour |
| | | (1 mg/mL) | | |

The Alzheimer's disease model mouse is an APP-PS1 trans-genic mouse model of AD which carries mutations for APP and presenilin-1 (APPswe and PSEN1dE9, respectively) resulting in increased Aβ production. Animals were injected with AP₂₂-POs and sacrificed after 1 hr or 24 hr. Aβ and tau levels in the brain and blood were measured by Western Blot and ELISA. The brains of mouse groups 1, 3 and 4 were also imaged via PET, using [18F](E)-4-(2-(6-(2-(2-(2-18F-fluoroethoxy)ethoxy)ethoxy)pyridin-3-yl)vinyl)-N-methylbenzamine as the radiolabel.

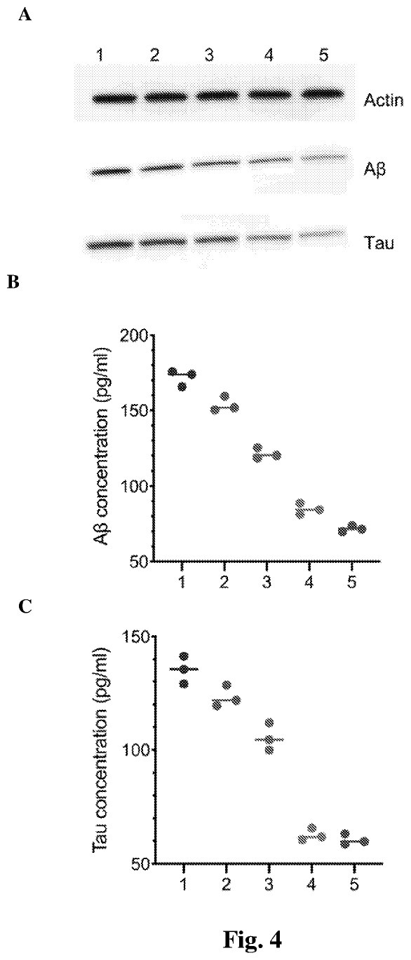

The Western blots in FIG. 4A show that in the diseased mice (groups 1 to 3), both amyloid-β and tau levels in the brain are reduced after polymersome administration, and that a greater reduction in these protein levels is observed when a larger amount of polymersomes are used. Even in the healthy mice (groups 4 and 5), which show much lower starting levels of amyloid-β and tau proteins, a decrease in the amount of these proteins is apparent after dosing with the polymersomes. The results are also tabulated graphically in FIG. 4B (for amyloid-β levels) and FIG. 4C (for tau levels).

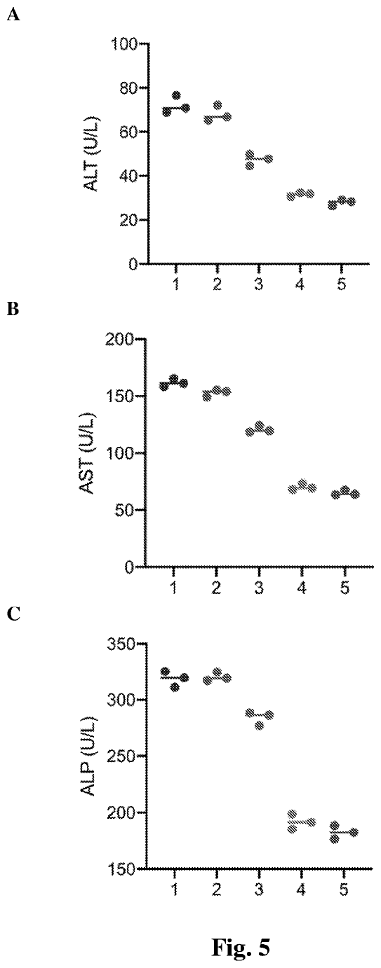

FIGS. 5A-5C show the levels of three liver function markers, alanine aminotransferase (ALT), aspartate aminotransferase (AST) and alkaline phosphatase (ALP), present in the different cohorts of mice. After treatment of diseased animals with the polymersomes (groups 2 and 3) there is an increase in each of these liver function markers, which is also observed in the PBS-treated diseased mice (group 1). This suggests that the Alzheimer's diseased mice have inherently higher levels of these markers. When healthy mice are treated with the polymersomes (group 5), no increase in the liver function markers is observed compared with the non-treated healthy mice (group 4).

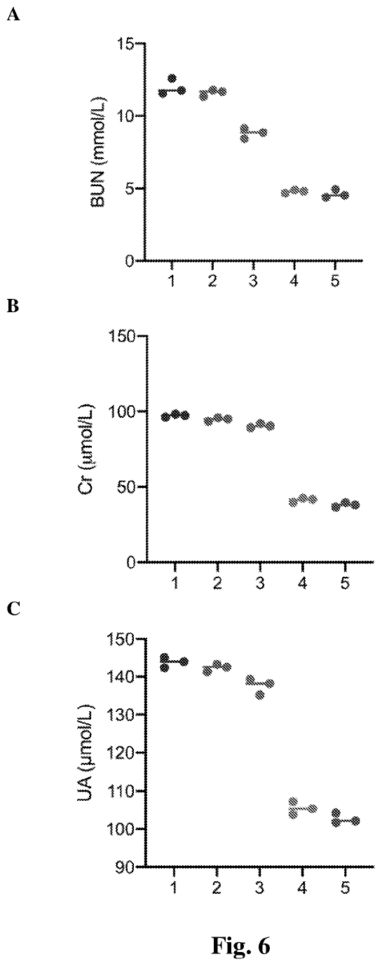

FIGS. 6A-6C show the level of three kidney function markers, blood urea nitrogen (BUN), creatine (Cr) and uric acid (UA), present in the different cohorts of mice. Similar results are observed to the liver function tests. After treatment of diseased animals with the polymersomes (groups 2 and 3) there is an increase in each of the kidney function markers, but this is also observed in the PBS-treated diseased mice (group 1). This suggests that the Alzheimer's diseased mice have inherently higher levels of these markers. When healthy mice are treated with the polymersomes (group 5), no increase in the kidney function markers is observed compared with the non-treated healthy mice (group 4).

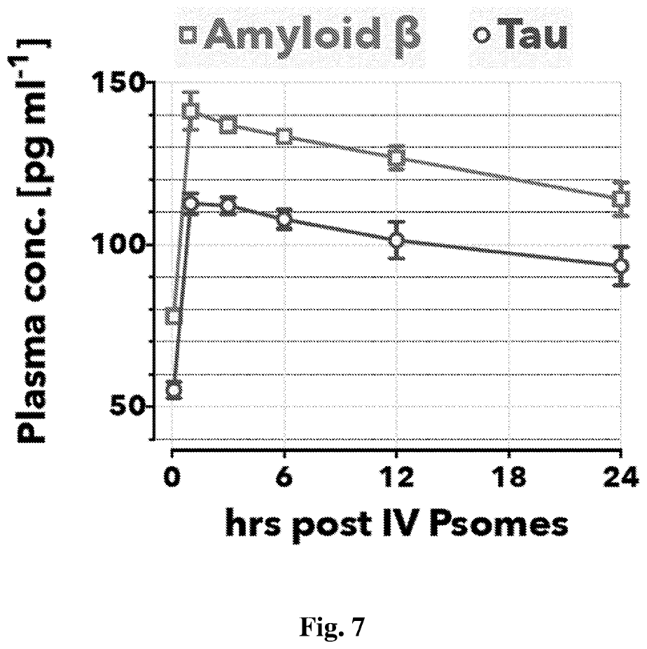

FIG. 7 shows the levels of amyloid beta and tau in blood plasma at various time points after the administration of polymersomes to Alzheimer's diseased mouse group 3. A rapid increase in plasma concentrations of amyloid beta and tau can be observed shortly after administration of the polymersomes, indicating transport of amyloid beta and tau from deposits in the brain into the blood plasma. The plasma levels of amyloid beta and tau then reduce over time due to clearance.

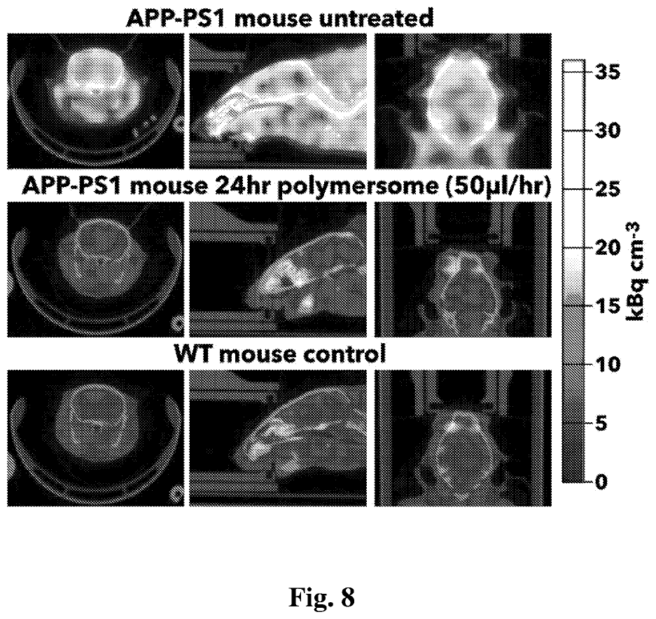

FIG. 8 shows PET scans of the brains of the mice in groups 1 (top), 3 (middle) and 4 (bottom). The scans show a significant reduction in the amount of amyloid beta present in the brain in the group of animals that was treated with polymersomes, when compared with the diseased animals.

1-22. (canceled)

23. A method for reducing amyloid-β and/or tau levels in an organ of a patient in need thereof, wherein said method comprises administration to said patient of a therapeutically effective amount of a nanoparticle or microparticle that comprises a ligand type on its external surface which is capable of binding to low density lipoprotein receptor-related protein 1 (LRP-1) on the surface of an endothelial cell, and thereby promoting transport of LRP-1 across said endothelial cell.

24. The method according to claim 23, wherein the organ is the brain and the endothelial cell is a brain endothelial cell.

25-26. (canceled)

27. A nanoparticle or microparticle for binding to the surface of an endothelial cell comprising:
(i) a ligand type on its external surface which is capable of binding to low density lipoprotein receptor-related protein 1 (LRP-1) on the surface of an endothelial cell, thereby promoting transport of LRP-1 across said endothelial cell; and
(ii) an encapsulated drug selected from an anti-Alzheimer's drug and/or a drug that is useful in reducing amyloid-β and/or tau levels or inhibiting amyloid-β and/or tau formation, preferably wherein said drug is selected from donepezil, galantamine, rivastigmine and memantine.

28. A nanoparticle or microparticle according to claim 27, wherein the endothelial cell is a brain endothelial cell.

29. A pharmaceutical composition comprising a plurality of the nanoparticles or microparticles according to claim 27, and one or more pharmaceutically acceptable excipients.

30. The method according to claim 23, wherein the transport of LRP-1 across the endothelial cell occurs via transcytosis, preferably wherein the transcytosis mechanism comprises transport of LRP-1 in structures that are stabilized by syndapin-2.

31. The method according to claim 23, wherein the nanoparticle or microparticle comprises from 2 to 1000 ligands of the ligand type, preferably from 5 to 500 ligands of the ligand type, more preferably from 10 to 200 ligands of the ligand type, yet more preferably from 15 to 100 ligands of the ligand type, and most preferably from 20 to 50 ligands of the ligand type.

32. The method according to claim 23, wherein the nanoparticle or microparticle is a polymersome, liposome, synthosome or micelle, and is preferably a polymersome.

33. The method according to claim 23, wherein the nanoparticle or microparticle comprises a polymer brush on its external surface.

34. The method according to claim 23, wherein the ligand type which is capable of binding to LRP-1 is Angiopep-2.

35. The method according to claim 23, wherein the nanoparticle or microparticle comprises a number of the ligand type (λ*A) such that the nanoparticle or microparticle satisfies the following relationship:
$$ln [ {( 1 + λ {Ae}^{- β ( {ϵ}_{B} + {U}_{S} )} )}^{ζ} - 1 ] ∈ [ 20 , 40 ]$$
wherein:
λ is the density of the ligand type on the external surface of the microparticle or nanoparticle (number per nm²);
A is the microparticle or nanoparticle surface area (in nm²);
ξ=ΓA where Γ is the LRP-1 surface density (number per nm²) and A is as defined above;
β=(k_{B}T)⁻¹ wherein k_{B} is the Boltzmann constant (in JK⁻¹) and T is the absolute temperature (in K);
εB is the single energy of binding of a ligand type/LRP-1 receptor pair (in J); and
uS is the steric potential between the nanoparticle or microparticle and the cell surface (in J).

36. The method according to claim 23, wherein the nanoparticle or microparticle comprises at least one further ligand type on its external surface that is capable of binding to a further receptor type on the cell surface.

37. The method according to claim 36, wherein:
(a) the nanoparticle or microparticle comprises from one to six further ligand types on its external surface, preferably from two to four further ligand types, and most preferably three further ligand types, wherein each ligand type is capable of binding to a complementary receptor type on said cell surface; or
(b) the at least one further ligand type is capable of binding to a receptor type selected from scavenger receptor class B, member 1 (SCARB1), a transferrin receptor (TFRC), folate receptor 1 (FOLR1) and epidermal growth factor receptor (EGFR); or
(c) the at least one further ligand type is selected from poly(2-(methacryloyloxy)ethyl phosphorylcholine), folic acid, transferrin, transferrin mimic peptide, and YHWYGYTPQNVI peptide.

38. The method according to claim 23, wherein the method is a method of treating or preventing Alzheimer's disease in the patient.

39. The method according to claim 23, wherein the method is a method of treating or preventing cerebral angiopathy in the patient.

40. The method according to claim 23, further comprising a drug encapsulated within the nanoparticle or microparticle, optionally wherein the drug is selected from an anti-Alzheimer's drug, a drug for treating cerebral angiopathy and/or a drug that is useful in reducing amyloid-β and/or tau levels or inhibiting amyloid-β and/or tau formation, preferably wherein the drug is selected from donepezil, galantamine, rivastigmine and memantine

41. The method according to claim 23, wherein the polymer brush comprises poly(ethylene glycol) (PEG), poly(vinyl pyrrolidone) (PVP), poly(2-methacryloyloxyethyl phosphorylcholine) (PMPC), poly(glycerol)s, poly(sulfobetaine), poly(carboxybetaine), poly(amino acid)s, polysarcosine, poly(2-oxazoline)s, poly(N-(2-hydroxypropyl)methacrylamide), polyglycols, heparin, dextran, poly(ethylene glycol)-poly(2-(diisopropylamino)ethyl methacrylate) and/or poly(oligo(ethylene glycol) methyl ether methacrylate) (POEGMA), optionally wherein the nanoparticle or microparticle comprises, on its external surface, a polymer brush comprising poly(ethylene glycol)-poly(2-(diisopropylamino)ethyl methacrylate) and a ligand type which is capable of binding to LRP-1.

42. The method according to claim 23, wherein each ligand on the external surface of the nanoparticle or microparticle is covalently bound to a poly(ethylene glycol) molecule.

43. The method according to claim 23, wherein the binding of the nanoparticle or microparticle to the endothelial cell surface results in an increase in the expression of LRP-1 in said endothelial cell.

44. A method for reducing amyloid-β and/or tau levels in an organ of a patient in need thereof, wherein said method comprises administration to said patient of a therapeutically effective amount of a pharmaceutical composition, wherein said composition comprises (i) a plurality of nanoparticles or microparticles that comprise a ligand type on their external surface which is capable of binding to low density lipoprotein receptor-related protein 1 (LRP-1) on the surface of an endothelial cell, and thereby promoting transport of LRP-1 across said endothelial cell, and (ii) one or more pharmaceutically acceptable excipients.


SEQUENCE LISTING (2 sequences)

SEQ ID NO: 1 - protein, 19 aa, synthetic construct
TFFYGGSRGKRNNFKTEEY

SEQ ID NO: 2 - protein, 12 aa, synthetic construct
YHWYGYTPQNVI